(19) United States
(12) Patent Application Publication
(10) Pub. No.: US 2026/0131172 A1
(43) Pub. Date: May 14, 2026

(54) METHOD AND APPARATUS FOR TREATMENT OF PULMONARY CONDITIONS

(71) Applicant: AerWave Medical, Inc., New York, NY (US)

(72) Inventors: Gerard J. Criner, Bryn Mawr, PA (US); Reinhard J. Warnking, Westlake, FL (US); Wei Wang, East Setauket, NY (US)

(73) Assignee: AerWave Medical, Inc., New York, NY (US)

(21) Appl. No.: 19/118,812

(22) PCT Filed: Oct. 6, 2023
(86) PCT No.: PCT/US2023/034610

§ 371 Date: Apr. 7, 2025

Related U.S. Application Data
(60) Provisional application No. 63/416,089, filed on Oct. 14, 2022.

(51) Int. Cl. A61N 7/02; A61B 17/00; A61B 17/12; A61B 17/32; A61B 90/00; A61N 7/00

(52) U.S. Cl. CPC A61N 7/022; A61B 17/1204; A61B 17/12104; A61B 17/12136; A61B 17/320068; A61B 2017/00398; A61B 2017/320069; A61B 2017/32007; A61B 2090/064; A61B 2217/005; A61N 2007/003; A61N 2007/0052

(57) ABSTRACT

Apparatus and methods for deactivating bronchial nerves, ablating smooth muscle, ablating goblet cells or performing lung volume reduction in a mammalian subject to treat asthma and COPD. An ultrasonic transducer is inserted into the bronchus as, for example, by advancing the distal end of a catheter bearing the transducer into the bronchial section to be treated. The ultrasonic transducer emits ultrasound so as to heat tissues throughout a relatively large impact volume as, for example, at least about 1 cm3 encompassing the bronchus to a temperature sufficient to inactivate nerve conduction, ablate smooth muscle, ablate goblet cells or remodel emphysematous tissue but insufficient to cause rapid ablation or necrosis of the surrounding healthy tissues.

### BACKGROUND OF THE INVENTION

This application claims priority to provisional application Ser. No. 63/416,089, filed Sep. 13, 2023, the entire contents of which are incorporated herein by reference.

### FIELD OF THE INVENTION

This invention relates to apparatus and methods for the treatment of emphysematous pulmonary conditions.

### BACKGROUND

Successful treatment of pulmonary diseases such as emphysema, asthma and COPD is important since these diseases represent a significant global health issue with reduced quality of life. While drug therapy (Bronchodilators, Anti Inflammatories and Leukotrines Modifiers) can be used, it is not always successful and is very expensive.

In approximately 30-40% of patients with COPD, their major structural phenotype is characterized by emphysematous tissue with damaged alveoli which leads to limited oxygen exchange and hyperinflation. Segmental lung volume reduction through implanted valves or coils or surgery is one treatment option. In other patients with COPD, mucous hypersecretion is a significant problem that contributes to increased morbidity and mortality; at present this is a significant unmet clinical need since there are no currently available effective therapies.

Asthma is a disorder that is characterized by airway constriction and inflammation resulting in breathing difficulties. Wheezing, shortness of breath and coughing are caused by increased mucus production, airway inflammation, smooth muscle contraction resulting in airway obstruction. This obstruction can be treated by injuring and scarring the bronchial walls. This remodeling of the bronchial walls stiffens the bronchi and reduces contractility. Mechanical means and heat application have been proposed as in U.S. Pat. No. 8,267,094 B2. Other approaches focus on destruction of smooth muscle cells surrounding the bronchia as described in U.S. Patent Application Publication No. 2012/0143099A1 and U.S. Pat. No. 7,906,124B2. Others describe applying RF energy to the bronchial wall and thereby directly widening the bronchi through a process which is disclosed in U.S. Pat. No. 7,740,017B2 and U.S. Pat. No. 8,161,978B2. Whatever the process, the bronchial wall will be damaged, and the procedure therefore must be performed in stages to limit damage and side effects as described in U.S. Pat. No. 7,740,017B2. European U.S. Pat. No. 2,405,841 describes applications of heat shocks through infused agents. Inactivating conduction of the nerves surrounding the bronchi has been proposed in U.S. Patent Publication No. 2012/0203216A1 through mechanical action i.e., puncturing, tearing, cutting nerve tissue.

It has been proposed to ablate nerve tissue by applying energy (RF, HIFU, Microwave, Radiation and Thermal Energy) directly to the nerves percutaneously. Such proposals do not explain how one is to identify the nerve location to align the energy focal zone (i.e., HIFU) with the nerve location. This is an issue since nerves are too small to be visualized with standard ultrasound, CT, or MRI imaging methods. Therefore, the focal zone of the energy field cannot be predictably aligned with the target or nerve location. U.S. Pat. No. 8,088,127B2 teaches to denervate by applying RF energy to the bronchial wall with the catheter positioned inside the bronchial lumen. It is proposed to protect the bronchial wall through simultaneous cooling of the wall. This of course makes the device structure complicated and bulky and therewith difficult to deliver through a bronchoscope working channel. Also, the RF ablation is limited to the electrode contact area which requires adding together several energy applications to create a circumferential treatment volume. Numerous ablation sectors need to be pieced together to obtain a circumferential ablation zone with increased probability of affecting nerves. Due to catheter size and the need for multiple energy applications per bronchus, denervation with a cooled RF ablation device is practically limited to denervation in the left and right main bronchi to keep the overall number of energy applications low (at least 4 per bronchus) and the procedural time acceptable. However, this main bronchial location carries the risk of esophageal and peri esophageal nerve damage which complicates the procedure further by causing gastroparesis, requiring fluoroscopic monitoring of the distance between ablation and an esophageal marker-balloon. How to safely simplify lung denervation procedures by employing circumferential ultrasound in secondary bronchi is described in U.S. Patent Application Publication No. 2021/0316161.

The sectorial RF ablation in main bronchi is not only complicated and time consuming but often also limited as far as efficacy is concerned because often RF energy delivery needs to be limited by reducing RF power or lesion geometry i.e., forgoing posterior ablation segments to avoid damaging peri esophageal nerves or the esophagus located in the vicinity of the posterior main bronchi. How to safely simplify lung denervation procedures by employing circumferential ultrasound in secondary bronchi is described in US Patent Application Publication No 2021/0316161. However, there is a need for a device and method to increase efficacy and ease of use further. That is, one can increase effectiveness and facilitate such procedures by performing lung denervation and smooth muscle and goblet cell ablation with one and the same treatment catheter in one procedure. It would be advantageous to reduce current multiple treatments such as described in U.S. Pat. No. 7,740,017B2 and the Alair System description, BSX. Reduction to a one-time treatment is much better tolerated by the patient.

There is a further need to provide apparatus and associated techniques for treating emphysematous tissue generally not otherwise implicated in asthma and COPD.

To explain the difficulties associated with accomplishing this task without causing other damage, the anatomy of the bronchial system, smooth muscle, nerves, and emphysematous tissue will be described now. Shown in FIG. 6 is an illustration of the bronchial tree with an emphysematous upper left lobe (16). FIG. 3 shows a cross section of a bronchial tube surrounded with smooth muscle (15) and a fluid filled treatment catheter (11) inserted. In addition, FIG. 5 shows a longitudinal section of a bronchus and the adjacent nerves (6). As can be seen, the bronchial nerves (6) surround the bronchial tubes. Different individuals have the nerves (6) in different locations around the bronchial tubes. Thus, the nerves may be at different radial distances from the central axis where the energy emitter (11) is placed (FIG. 3). The nerves also may be at different locations around the circumference of the bronchial tubes. It is not practical to locate the bronchial nerves by referring to anatomical landmarks. Moreover, it is difficult or impossible to locate individual bronchial nerves using common in vivo imaging technology.

The inability to locate and target the bronchial nerves makes it difficult to disconnect the bronchial nerve activity using non-surgical techniques without causing damage to the bronchial walls or causing other side effects. For example, attempts to apply energy to the bronchial nerves can cause effects such as stenosis, and necrosis of adjacent tissues. In addition, the inability to target and locate the bronchial nerves makes it difficult to ensure that bronchial nerve activity has been discontinued enough to achieve an acceptable therapeutic result.

U.S. Pat. No. 8,088,127B2 suggests the use of a radio frequency (“RF”) emitter connected to a catheter, which is inserted in the bronchial tree. The RF emitter is placed against the bronchial wall and the RF energy is emitted to heat the nerves to a temperature that reduces the activity of bronchial nerves which happen to lie in the immediate vicinity of the emitter. To treat all the nerves surrounding the bronchial tubes, the RF emitter source must be repositioned around the inside of each bronchial tube section multiple times. To protect the bronchial wall, this RF heat application is combined with a cooling application which makes the procedure more complicated. The emitter may miss some bronchial nerves, leading to an incomplete treatment. Moreover, the RF energy source (electrode) must contact the bronchial wall to be able to heat the surrounding tissue and nerves, which may cause damage or necrosis to the inner lining of the bronchi. Since each denervation consists of several segmental RF applications and the cooled RF treatment catheter is rather bulky, RF denervation is practically limited to main bronchial locations. This in turn, due to the vicinity of the esophagus, requires safety measures like placement of an esophageal marker balloon and fluoroscopic imaging to monitor the distance between marker and treatment balloons. If a safe distance between marker and treatment balloons cannot be achieved, ablation energy needs to be limited or posterior ablation segments need to remain untreated to protect the esophagus and peri esophageal nerves from damage.

With a circumferential ultrasound catheter, a single application of a 360-degree ablation zone is accomplished so that more distal treatment sites can be chosen without increasing the number of energy applications to impractical levels, as described in U.S. Patent Application Publication No. 2021/0316161 for denervation in secondary bronchi.

It would be advantageous to provide further applications utilizing single energy application per bronchus.

### SUMMARY OF THE INVENTION

The present invention overcomes the problems and deficiencies of the prior art.

As described hereinafter, the present invention proposes in some embodiments to perform lung volume reduction through thermal and mechanical ultrasound remodeling of emphysematous tissue and to treat mucous hypersecretion through goblet cell ablation.

Ini accordance with one aspect of the present invention, denervation in the lobar central airways is realistic and practical since only 1 energy application per bronchus is required compared to at least 4 with RF segmental ablation techniques. The present invention thus ensures a more uniform energy application to create more efficient and efficacious nerve ablation with a significantly easier process and a shorter procedural time that significantly improves the efficacy and safety of the procedure.

One aspect of the present invention is directed to apparatus and associated methodology for the treatment of emphysematous lung tissue. The invention contemplates ultrasonic thermal and mechanical remodeling of emphysematous tissue by application of ultrasonic energy to the tissue. An ultrasound treatment catheter can be advanced into an emphysematous tissue section (16 in FIG. 6) to perform segmental lung volume reduction by inducing inflammation and subsequently a focal fibrotic reaction in emphysematous tissue that strategically reduces lung volume in patients suffering from hyperinflation. This non-surgical, endoscopic segmental lung volume reduction stimulates the body's natural healing processes in response to ultrasound heating and cavitation effects, without leaving any implanted materials inside the treated lung section.

Other aspects of the invention are directed to an apparatus for inactivating bronchial nerve conduction, performing smooth muscle ablation, and optionally goblet cell ablation, preferably in third and fourth generation bronchi. The ultrasound treatment volume is optimized through energy-application time, power and cooling temperature adjustments for such procedures. Thus, the apparatus includes an ultrasound transducer adapted for insertion into the bronchial system of the mammalian subject exemplarily through a bronchoscope working channel. The ultrasound transducer preferably is arranged to transmit circumferential ultrasound energy. The apparatus preferably also includes an actuator or control unit (e.g., programmed microprocessor or hard-wired logic circuit) electrically connected to the transducer. The actuator preferably is configured to control through time and power variations the ultrasound transducer to transmit ultrasound energy into a circumferential impact volume encompassing a target bronchial branch or tube so that the ultrasound energy is applied at a therapeutic level sufficient to inactivate conduction of bronchial nerves and/or ablate smooth muscle and goblet cells or remodel emphysematous tissue throughout the impact volume. Given the wide variation of bronchial diameters and different depths of nerves, smooth muscle, goblet cells and emphysematous tissue, this procedure is achievable by combining the therapeutic operation with a diagnostic mode, e.g., a volumetric A mode, to determine the diameter and adjust the therapeutic ultrasound energy (time, power, and water-cooling temperature) accordingly as shown in FIG. 7.

The apparatus may further include a catheter with a distal end and a proximal end, the transducer being mounted to the catheter adjacent the distal end. The transducer can be constructed and arranged inside a compliant balloon which when inflated contacts the bronchial wall. This compliant balloon is filled with a circulating cooling and coupling fluid to conduct ultrasound energy from the transducer to the bronchial walls and surrounding tissue and nerves. This cooling fluid also transports excessive heat away from the transducer and protects the inner bronchial lining from injury. About half of the electrical energy supplied to the transducer is converted into heat while the other half is converted to ultrasonic energy. The transducer is adapted to transmit the ultrasound energy in a 360° cylindrical pattern surrounding a transducer axis, and the catheter may be constructed and arranged to hold the axis of the transducer generally parallel to the axis of the bronchial tube.

The ultrasound transducer is not only configured to emit therapeutic ultrasound but also to work in a diagnostic mode to generate a volume integrated A mode signal from throughout the treatment volume which is analyzed to (i) ensure circumferential coupling, (ii) measure the mean bronchial diameter to optimize dosing (power, time) for denervation and or smooth muscle and goblet cell ablation or emphysematous tissue ablation, and/or (iii) ensure inter cartilage positioning as described in U.S. patent application Ser. No. 17/350,848, Publication No. 2021/0316161. By analyzing the typical echo pattern of emphysematous tissue in the volumetric A mode signal, the treatment window (FIG. 7) can be optimized by adjusting time, power and cooling temperature to overlay the emphysematous tissue volume.

A further parameter to adjust the treatment volume with the target tissue is pre-cooling of the bronchus and surrounding tissue which moves the treatment volume further outwards. After the emphysematous tissue has been localized through its volumetric A mode signal characteristic, it is flooded with a saline solution through a catheter lumen in order to ensure therapeutic ultrasound penetration throughout the emphysematous tissue volume(s). To ensure selective flooding of the emphysematous lung segment to be treated, the balloon in its expanded state confines the saline in a distal treatment section. After the ultrasound treatment a vacuum can be applied to extract saline and mucous from the treated lung section.

A method according to a denervation aspect of the invention preferably includes the steps of inserting an ultrasound transducer into an airway of second generation of the subject and actuating the transducer to transmit therapeutically effective ultrasound energy into a circumferential impact volume encompassing the bronchial branch. The ultrasound energy preferably is applied so that the therapeutically effective ultrasound energy inactivates conduction of all the nerves in the impact volume, typically at a few mm depth from the bronchial inner lining. For example, the step of actuating the transducer may be to maintain the temperature of the bronchial wall below 45° C. while heating the solid tissues including the nerves to about 60 C. In another application, smooth muscle will be targeted and the impact volume adjusted to encompass very shallow depth of about 1 to about 2 mm from the inner bronchial wall. For lung volume reduction, a large depth will be selected based on the diagnostic mode measurement of the emphysematous tissue depth characterized in the volumetric A mode signal by its frequency content. Mucous cell hyperplasia will be targeted by adjusting the treatment volume (FIG. 7) into airway epithelial layers by reducing power and time accordingly and increasing the coolant temperature up to body temperature levels.

The treatments can be performed without measuring the temperature of energy source and adjacent tissues as described in Athmatx patents. Based on the bronchial diameter measurement, the therapeutic dose (power, time, cooling temperature and precooling time) may be optimized for each anatomical situation. Moreover, the treatment preferably is performed without causing injury to the mucosa. The preferred methods and apparatus of the present invention can inactivate relatively long segments of smooth muscle, goblet cells, bronchial nerves, and/or emphysematous tissue, to reduce procedure time.

In accordance with the present invention, ultrasonically mediated denervation in the lobar central airways is realistic and practical since only 1 energy application per bronchus is required compared to at least 4 applications with RF segmental ablation techniques. The ultrasonic treatment of the present invention thus ensures more uniform energy application to create more efficient and efficacious neural and smooth muscle ablation with a significantly easier and shorter procedural time that significantly improves the efficacy and safety of the procedure.

The present invention in some embodiments, may include an apparatus adapted to generate pulsed ultrasonic standing waves in emphysematous alveoli, to induce controlled amounts of mechanical stress in the emphysematous damaged alveoli and thereby break up or modify emphysematous tissue, causing inflammation, and resulting in remodeling of alveoli structure. Alternatively or additionally, as discussed above, the apparatus of the invention implements bronchial denervation and smooth muscle ablation to maximize aeration of the alveoli. The ultrasound generating componentry of the apparatus may serve to denervate and ablate smooth muscle tissue at least in the third and fourth generation bronchial airways. In addition, the present invention contemplates goblet cell ablation in the bronchial passageways treated.

Multipurpose apparatus for remodeling emphysematous tissue in a mammalian subject comprises, in accordance with one aspect of the present invention, a catheter or other tubular member adapted for insertion into the bronchial tree of the mammalian subject so that a distal end of the catheter is disposable in a third or fourth generation bronchus, the catheter being attachable at a proximal end to a source of pressurized or pressurizable liquid, and the catheter having a lumen and a port opening proximate the distal end of the catheter and communicating with the source of pressurized or pressurizable liquid for injecting liquid (e.g., saline) into the bronchial tree distally of the distal end of the catheter. The apparatus in this aspect can also comprise an alternately expandable and contractible occlusive member adapted for insertion in a collapsed configuration into the bronchial tree of the mammalian subject and subsequently expandable in the third or fourth generation bronchus to trap liquid in a bronchial tree section distally of the expanded occlusive member. The apparatus in this aspect can additionally comprise an acoustic transducer inside a non-compliant cooling balloon with circulating non-ionic cooling fluid and adapted for (a) insertion into the bronchial tree of the mammalian subject and disposable in wave-transmitting contact with the trapped liquid, via the balloon and the cooling liquid therein, and (b) emitting acoustic (sub-ultrasonic) and/or ultrasound energy into the bronchial tree distally of the expanded occlusive member. An actuator or control unit (such as a programmed microprocessor or a hard-wired digital circuit) is electrically connected to the transducer, the actuator or control unit being adapted to activate or energize the transducer and transmit acoustic and/or ultrasound energy to emphysematous tissue via the trapped liquid, with the energy being applied at a therapeutic level sufficient to damage the emphysematous tissue and thereby initiate autogenic remodeling of the emphysematous tissue.

According to a feature of the present invention, the actuator or control unit can be adapted to control the transducer to transmit the acoustic and/or ultrasound energy in pulses to mechanically remodel the emphysematous tissue by inducing mechanical stress in the affected alveoli segments, thereby inducing remodeling thereof. More particularly, the actuator or control unit provides the transducer with pulses of selected duration, frequency, and inter-pulse spacing to so pulse the energy to induce mechanical stress in the emphysematous tissue of the subject and thereby break up or modify emphysematous damaged alveoli and induce remodeling of alveoli structure.

The actuator or control unit can be configured to energize the transducer with low-power pulses and to process incoming ultrasonic echoes to determine size and structure of the emphysematous tissue sections. The actuator or control unit may be further configured to select parameters of pulse duration, frequency, and inter-pulse spacing in accordance with an average alveoli size and structure as determined by A mode frequency analysis of the emphysematous tissue section.

Pursuant to a further feature of the present invention, the actuator or control unit can be configured to energize the transducer to emit test pulses into the trapped liquid and to analyze incoming ultrasonic echoes received in response to the test pulses to determine resonance characteristics of the emphysematous tissue section containing the trapped liquid distally of or around the expanded occlusive member. The actuator or control unit can be further configured to select parameters of pulse duration, inter-pulse spacing and pulse frequency in accordance with determined resonance characteristics.

The actuator or control unit may be more particularly configured to energize the transducer to emit multiple test pulses of different combinations of pulse duration, pulse frequency and inter-pulse spacing in an iterative process to optimize the selected parameters of pulse duration, pulse frequency and inter-pulse spacing.

The invention thus contemplates apparatus for emitting acoustic and/or ultrasound energy in pulses to mechanically remodel emphysematous tissue by inducing mechanical stress in affected alveoli segments, thereby prompting remodeling thereof.

Pursuant to an embodiment of the system of the present invention, first and second catheters are provided. The second catheter has a distal end and proximal end, and the transducer is mounted to the second catheter, inside a non-compliant cooling balloon holding circulating non-ionic fluid adjacent the distal end of the second catheter. The second catheter may be movable independently of, e.g., within, the first catheter. Alternatively, in the case of only one catheter, the transducer may be mounted to a distal end of the catheter inside the occlusion balloon which is being positioned inside the emphysematous tissue volume.

Preferably, the occlusive member of the present invention comprises a compliant and alternately collapsible and expandable balloon exemplarily attached to the one, or the first, catheter at the distal end thereof. The balloon may include a porous membrane to engage the wall of the bronchus with fluid for facilitating adjustments in the position of the balloon along the bronchus. The transducer for pulsed treatment is disposed inside a balloon filled with non-ionic cooling liquid; the pulses are conducted into the trapped liquid (typically a saline solution) via the coupling liquid and the balloon.

Where the apparatus includes components for bronchial denervation, smooth muscle ablation and goblet cell ablation, particularly but not exclusively of the third and fourth generation bronchi, the actuator or control unit may be configured to control the transducer to transmit ultrasound energy into an impact volume encompassing the third or fourth generation bronchus so that the ultrasound energy is applied at a therapeutic level sufficient to inactivate conduction of bronchial nerves, ablate smooth muscle and ablate goblet cells throughout an impact volume surrounding the third or fourth generation bronchus.

The apparatus of the present invention can enable removing the trapped liquid and mucous after application of the ultrasound energy to damage emphysematous tissue and initiate autogenic remodeling. The catheter used to inject the ultrasound-carrying liquid may be connected at its proximal end to a suction source. A suction pressure-sensor may be provided to detect obstruction of the catheter, the catheter being then pressurized for a short time to eject the obstruction and enable continued evacuation of the liquid in the section of the bronchial tree under treatment. This feature will also enable mucous removal from an emphysematous lung section.

A method for treating emphysematous tissue comprises, in accordance with one aspect the present invention, (i) inserting a distal portion of an elongate introducer instrument into a bronchial tree of a subject diagnosed as having at least one emphysema-damaged lung region so that a distal end portion of the elongate introducer instrument is disposed in a bronchus proximate or in the at least one emphysema-damaged lung region, (ii) injecting a liquid into the bronchial tree via the elongate introducer instrument, (iii) either before or after, but preferably after, step ii, expanding an occlusive member disposed at the distal end portion of the elongate introducer instrument to trap the injected liquid in a section of the bronchial tree including the emphysema-damaged lung region, (iv) inserting, via the elongate introducer instrument, an acoustic/ultrasound transducer, (v) disposing the acoustic/ultrasound transducer contained in a cooling balloon in wave-transmitting contact with the liquid trapped in the section of the bronchial tree, and (vi) thereafter activating or energizing the ultrasound transducer to emit acoustic/ultrasound energy into the sealed or trapped liquid at a therapeutic level sufficient to damage emphysematous tissue and initiate autogenic remodeling of the emphysematous tissue.

As indicated above, the ultrasound transducer may be disposed inside a compliant expandable balloon filled with a circulating non-ionic cooling and coupling fluid. The actuator or control unit may be configured to control the temperature of the fluid, for instance, via a heating element and/or a coiling coil, so that the temperature induced in the smooth muscle and nerves by the application of ultrasound energy in the impact volume may be maximized, without unduly damaging the mucosa and wall of the bronchial tube at the locus of the treatment. The apparatus may include a temperature sensor, for instance, in the balloon, to ensure that the coupling fluid maintains a level of thermal energy that is safe for the bronchial tube tissue and mucosa.

Where the apparatus of this invention, utilizable for bronchial nerve, smooth muscle and goblet cell ablation as well as emphysematous tissue remodeling, includes a catheter provided at the distal end with the compliant balloon and the transducer mounted to the catheter adjacent the distal end, the compliant balloon is expandable by pressurizing the coupling fluid to contact the bronchial wall. The coupling fluid (non-ionic, non-conductive) is a circulating cooling fluid that serves in part to conduct ultrasound energy from the transducer to the bronchial walls and surrounding tissue and nerves. Pursuant to its cooling function, the coupling fluid transports excessive heat away from the transducer and protects the bronchial lining from injury. (About half of the electrical energy supplied to the transducer is converted into heat while the other half is converted to ultrasonic energy.) The transducer is adapted to transmit the ultrasound energy in a 360° cylindrical pattern surrounding a transducer axis, and the catheter may be constructed and arranged to hold the axis of the transducer generally parallel to the axis of the bronchial tube. The actuator or control unit is configured not only to activate the transducer to emit therapeutic ultrasound but also in certain embodiments to operate the transducer in a diagnostic mode to generate signals, e.g., a volumetric A mode signal, integrating ultrasound echoes throughout the treatment volume, which is analyzed to (i) ensure circumferential coupling, (ii) measure the mean bronchial diameter to optimize dosing (power, time), to analyze emphysematous tissue structures and in the case of denervation in secondary bronchi, and/or (iii) ensure inter cartilage positioning as described in U.S. Patent Publication No. 2021/0316161.

A method for pulsing ultrasound to ablate nerves or smooth muscle or goblet cells or remodel alveolar structure according to the present invention may additionally include the steps of inserting an ultrasound transducer into a bronchus of the subject and operating the actuator or control unit to activate the transducer to transmit therapeutically effective ultrasound energy into a circumferential impact volume encompassing the bronchus. The actuator or control unit controls the applied ultrasound energy to inactivate conduction of all the nerves in the impact volume and simultaneously or separately ablate smooth muscle, and optionally goblet cells, within the treatment volume. For example, the actuating of the transducer may be implemented so as to maintain the temperature of the bronchial wall below 45° C. while heating the solid tissues in particular airway smooth muscle (ASM) within the impact volume, including the bronchial nerves in the impact volume, to at least about 60° C. As discussed above, the actuator or control unit is preferably configured to maintain the temperature of the bronchial wall at a temperature below 45° C. in part by ensuring a sufficiently low temperature of the coupling fluid in the balloon.

Because the impact volume is relatively large, and because the tissues throughout the impact volume preferably reach temperatures sufficient to inactivate nerve conduction and cause smooth muscle ablation, the preferred methods according to this aspect of the invention can be performed successfully without precisely determining the actual locations of the bronchial nerves or smooth muscle fibers. The treatment can be performed without measuring the temperature of electrodes and adjacent tissues as described in the literature and patents to Athmatx. Based on the volumetric A mode bronchial diameter measurement, the therapeutic dose (power, time) will be optimized for each anatomical situation. Moreover, the treatment preferably is performed without causing injury to the mucosa. The present apparatus can be used to inactivate relatively long segments of smooth muscle and bronchial nerves, so as to reduce the possibility of nerve or smooth muscle recovery which would re-establish contraction along the inactivated smooth muscle segments and distal thereof.

The present invention also contemplates combined smooth muscle ablation and denervation in 3rd and/or 4th generation bronchial segments. The invention provides probes which can be used in the method and apparatus discussed herein, and apparatus incorporating components/features for performing the steps of the methods discussed herein.

The physical properties of ultrasound heating allow a miniaturized structure of the ultrasound catheter with the cylindrical energy source located at its center. This allows for the ultrasound catheter to not only perform denervation in proximal bronchi but also smooth muscle ablation in distal bronchi. Additionally, a smaller bronchoscope can be used that enhances operator performance and in turn procedural safety.

Pursuant to certain embodiments of the present invention, the ultrasound treatment catheter is advanced into emphysematous tissue regions (16 in FIG. 6) to perform segmental lung volume reduction. The application of ultrasonic waveform energy induces inflammation and subsequently a focal fibrotic reaction in emphysematous tissue that strategically reduces lung volume in patients suffering from hyperinflation. This non-surgical, endoscopic segmental lung volume reduction uses the body's natural healing processes in response to ultrasound heating, mechanical disruption, and/or cavitation effects without leaving any implanted materials inside the lung section.

Furthermore, the ultrasound treatment catheter can be advanced into bronchial sections with mucous hypersecretion adjusted to perform goblet cell ablation.

In addition, in certain embodiments, the ultrasound treatment volume can be optimized through time, power and cooling temperature adjustments for smooth muscle ablation. As discussed herein, ultrasonically mediated denervation in the lobar central airways is achievable and practical since only 1 energy application per bronchus is required compared to at least 4 applications with RF segmental ablation techniques. The ultrasonic treatment of the present invention ensures more uniform energy application to create more efficient and efficacious neural and smooth muscle ablation with a significantly easier and shorter procedural time that would significantly improve the efficacy and safety of the procedure.

### BRIEF DESCRIPTION OF THE DRAWINGS

So that those having ordinary skill in the art to which the subject invention appertains will more readily understand how to make and use the apparatus (device) disclosed herein, preferred embodiments thereof will be described in detail hereinbelow with reference to the drawings, wherein:

FIG. 1 is an anatomical view of typical main bronchial trunks and associated structures and an apparatus and system controlling ablation pursuant to the present invention.

FIG. 2 shows a treatment catheter of the present invention advanced through a bronchoscope into the right bronchial branch and includes a diagrammatic sectional view depicting a circumferential ultrasound treatment volume (in phantom).

FIG. 3 shows a cross section through a bronchial tube with smooth muscle with an ultrasound transducer of the catheter in FIG. 2 in the center surrounded by circulating cooling fluid in a compliant balloon.

FIG. 4 is partially a schematic longitudinal cross-sectional view of a distal end portion of a bronchial tree, partially a side elevational view and partially a block diagram of a system for treating emphysematous tissue pursuant to the present invention.

FIG. 5 shows a right bronchial branch with adjacent nerves running alongside the bronchial tree.

FIG. 6 shows a bronchial tree in its entirety with an emphysematous upper left lobe.

FIG. 7 is a graph of tissue temperature as a function of distance from an ultrasound transducer and time of transducer activation, providing an example for temperature profiles and resulting treatment depths determined by acoustic power and time and cooling temperature optimized in this case for a denervation treatment in a bronchus with a diameter of 10 mm.

### DETAILED DESCRIPTION

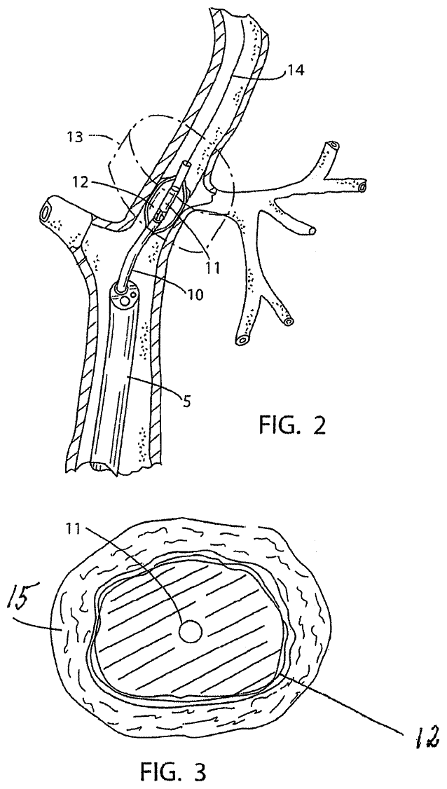

Referring now to the drawings and particular embodiments of the present disclosure, wherein like reference numerals identify similar structural features of the apparatus throughout the several views, an apparatus according to one embodiment of the invention is illustrated in FIG. 2. The apparatus is shown advanced through the working channel of a bronchoscope 5.

The apparatus includes a tubular member in the form of a catheter 10 having a proximal end, a distal end and a proximal-to-distal axis which is preferably coincident with the bronchial axis. The catheter can alternatively be advanced through a sheath or directly without any delivery instrument over a guide wire 14 which has been placed by electromagnetic navigation bronchoscopy (ENB). The sheath, generally, may be in the form of an elongated tube having a proximal end, a distal end and a proximal-to-distal axis. As used in this disclosure with reference to elongated elements for insertion into the body, the term “distal” refers to the end which is inserted into the body first, i.e., the leading end during advancement of the element into the body, whereas the term “proximal” refers to the opposite end which is closer to the clinician. The sheath may be a steerable sheath. Thus, the sheath may include known elements such as one or more pull wires (not shown) extending between the proximal and distal ends of the sheath and connected to a steering control arranged so that actuation of the steering control by the operator flexes the distal end of the sheath in a direction transverse to the axis. The scope can also be steerable.

Catheter 10 has a compliant balloon 12 mounted at the distal end. In its inflated condition (FIGS. 2 and 3), balloon 12 circumferentially engages the bronchial wall and therewith allows for ultrasound to be conducted from an ultrasound transducer 11 into the bronchial wall and surrounding tissues.

Transducer 11 is mounted adjacent the distal end of catheter 10 within balloon 12. Transducer 11, which is preferably formed from a ceramic piezoelectric material, is of a tubular shape and has an exterior emitting surface in the form of a cylindrical surface of revolution about the proximal-to-distal axis of the transducer 11. The transducer 11 typically has an axial length of approximately 2-10 mm, and preferably about 6 mm. The outer diameter of the transducer 30 is approximately 1.5-3 mm in diameter, and preferably about 2 mm. The transducer 11 also has conductive coatings (not shown) on its interior and exterior surfaces. Thus, the transducer may be physically mounted on a metallic support tube which in turn is mounted to the catheter. The coatings are electrically connected to ground and signal wires. Wires extend from the transducer 11 through a lumen in the catheter shaft to a connector electrically coupled with the ultrasound system. The lumen extends between the proximal end and the distal end of a catheter 10, while the wires extend from the transducer 11, through the lumen, to the proximal end of the catheter 10. Transducer 11 is arranged so that ultrasonic energy generated in the transducer is emitted principally from the exterior emitting surface. Thus, the transducer may include features arranged to reflect ultrasonic energy directed toward the interior of the transducer so that the reflected energy reinforces the ultrasonic vibrations at the exterior surface. For example, support tube and transducer 11 may be configured so that the energy emitted from the interior surface of the transducer 11 is reflected to enhance the overall efficiency of the transducer. In this embodiment, the ultrasound energy generated by the transducer 11 is reflected at an interior reflector to reinforce ultrasound energy propagating from the transducer 11, thereby ensuring the ultrasound energy is directed outwardly from an external surface of the transducer 11.

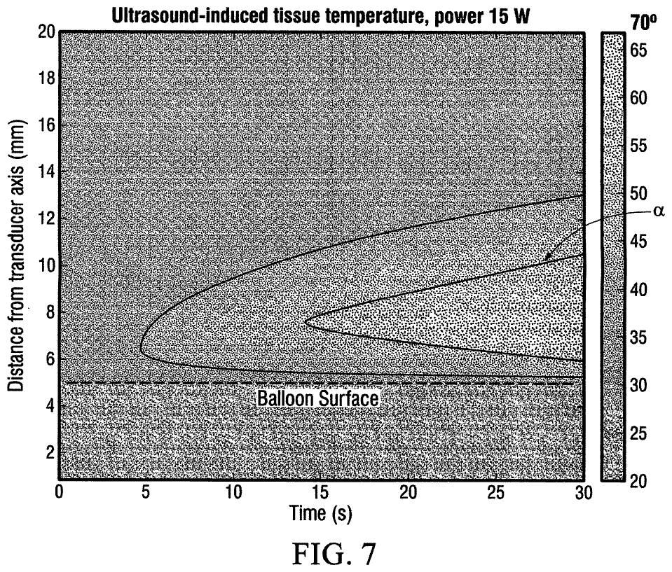

Transducer 11 is also arranged to convert ultrasonic echoes or waves impinging on the exterior surface into electrical signals on wires. If a reflecting structure such as a bronchial wall is not perfectly circular, the widths of the reflected signal will represent the difference between a maximum diameter of the reflecting structure, dmax, and a minimum diameter dmin. Stated another way, transducer 11 can act either as an ultrasonic emitter or an ultrasonic receiver. The receiving mode is of particular importance for optimizing the therapeutic impact volume through power, time and cooling temperature adjustments as shown in FIG. 7, based on interpretation of an integrated volumetric A mode signal.

It is to be noted that transducer 11 is operatively connected to an actuator or control unit 104 that provides both low-power diagnostic and high-power therapeutic electrical activation signals to the transducer, at different times. The actuator or control unit 104 may be programmed or hard-wired to calculate and select intensity and duration of outgoing therapeutic ultrasonic waveforms (as well as control of coupling fluid temperature). The actuator or control unit 104 may be additionally programmed or hard-wired to interpret diagnostic echoes, for instance, to determine resonance characteristics of distal end pulmonary structures including fourth generation bronchi and emphysematous alveolar structures.

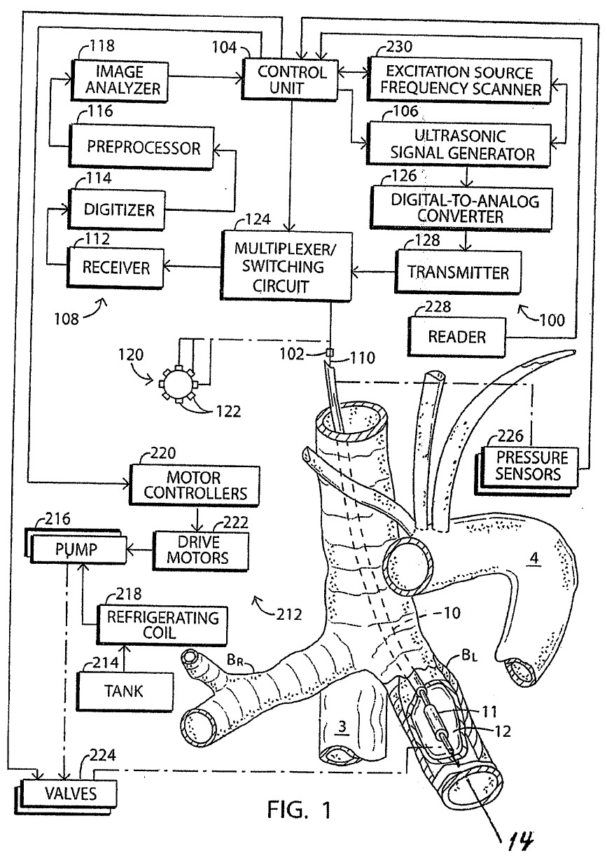

The transducer 11 is designed to operate, for example, at a frequency of approximately 1 MHz to approximately a few tens of MHz, and typically at approximately 10 MHz. The actual frequency of the transducer 11 typically varies somewhat depending on manufacturing tolerances. The optimum actuation frequency of the transducer may be encoded in a machine-readable or human-readable element (not shown) such as a digital memory, bar code or the like affixed to the catheter. Alternatively, the readable element may encode a serial number or other information identifying the individual catheter, so that the optimum actuation frequency may be retrieved from a central database accessible through a communication link such as the internet. Actuator or control unit 104 may be programmed or hard-wired, as discussed hereinafter, to activate transducer 11 in temporally spaced periods to generate pulsations to induce alveolar remodeling and optimize its effects based on diagnostic mode measurements and in some embodiments on AI analysis thereof. An ultrasound system including actuator or control unit 104 is releasably connected to catheter 10 and transducer 11 through a plug connector 102 (FIG. 1). The ultrasound system includes an ultrasound excitation source or ultrasonic signal or waveform generator 106 configured to control the amplitude and timing of the electrical signals so as to control the power level and duration of the ultrasound signals emitted by transducer 11 to optimize the therapeutic window for different applications, i.e., denervation, smooth muscle- and goblet cell-ablation or emphysematous tissue remodeling as indicated in FIG. 7. The excitation source is also arranged to detect electrical signals generated by transducer 11 and appearing on wires and communicate such signals to the control unit.

An energization circuit 100 including control unit 104 and ultrasonic signal generator 106 also includes a detection subcircuit 108 arranged to detect electrical signals generated by transducer 11 and transmitted via wires 110 and communicate such signals to the control unit 104. More particularly, detection subcircuit 108 includes a receiver or echo signal extractor 112, a digitizer 114, an ultrasonic echo signal preprocessor 116, and an image analyzer 118 connected in series to one another. Ultrasonic signal generator 106 produces both therapeutic denervation or tissue ablation signals and outgoing diagnostic A mode signals. As discussed hereinafter, the outgoing diagnostic signals and the returning echo signals may be transmitted and picked up by transducer 11. A multiplexer or switching circuit 124 is operated by control unit 104 to switch to a receiving mode after diagnostic signals are emitted during a transmitting mode via a digital-to-analog converter 126 and a transmitter module 128.

In the foregoing embodiments, the diagnostic mode is performed first, following by application of the therapeutic mode. In alternate embodiments, the therapeutic mode can be interleaved with the diagnostic mode and provided in a form of alternate patterns. For example, a therapeutic mode with pulsed signals can be applied intermittently with the diagnostic mode, at equal or unequal intervals, as desired, in a quasi-simultaneous method.

In some embodiments, the diagnostic mode can further be used to determine a tissue type or tissue state. That is, ultrasonic energy can be emitted at a sub-therapeutic level as described herein to assess.

As depicted in FIG. 1, a circulation device 212 is connected to lumens (not shown) within catheter 10 which in turn communicates with balloon 12. The circulation device 212 is arranged to circulate a liquid, preferably an aqueous liquid, through catheter 10 to transducer 11 in balloon 12. Circulation device 212 may include elements such as a tank 214 for holding the circulating coolant, pumps 216, a refrigerating coil 218, or the like for providing a supply of liquid to the interior space of the balloon 12 at a controlled temperature, preferably at or below body temperature. The control unit interfaces with the circulation device to control the flow of fluid into and out of the balloon 12, thereby effectuating balloon expansion and contraction. By lowering the coolant temperature, the inner radius of the circumferential treatment volume can be increased in order to protect certain structures like the inner bronchial lining from harmful temperatures. Control unit 104 interfaces with circulation device 212 to control the flow of fluid into and out of the balloon 12. For example, the control unit 104 may include motor control devices 220 linked to drive motors 222 associated with one or more pumps 216 for controlling the speed of operation of the pumps. Motor control devices 220 can be used, for example, where pumps 216 are positive displacement pumps, such as peristaltic pumps. Alternatively, or additionally, control unit 104 may include structures such as one or more controllable valves 114 connected in the fluid circuit for varying resistance of the circuit to fluid flow (not shown).

The ultrasound system may further include one or more pressure sensors 226 (FIG. 1) to monitor the liquid flow through the catheter 10. At least one pressure sensor 226 monitors the flow of the liquid to the distal end of catheter 10 to determine if there is a blockage while another pressure sensor 226 monitors leaks in the catheter 10. While the balloon 12 is in an inflated state, the pressure sensors 226 maintain a desired pressure in the balloon preferably so that the balloon occludes the bronchus under the regulation of control unit 104 pursuant to programmed analysis or processing by hard-wired circuitry of the volumetric A-mode signal.

A central lumen through catheter 10 allows for saline or suction to be applied to the lung section distal to the inflated balloon 12. This allows the clinician to flood distal lung sections to enable ultrasound propagation and therewith thermal and mechanical interaction with the emphysematous tissue and further permits the clinician to remove the saline and mucous after ultrasound treatment.

The ultrasound system incorporates a reader 228 for reading a machine-readable element on catheter 10 and conveying the information from such element to the control unit 104. As discussed above, the machine-readable element 228 on the catheter 10 may include information such as the operating frequency and efficiency of the transducer 11 in a particular catheter 10, and the control unit 104 may use this information to set the appropriate frequency and power for exciting the transducer. Alternatively, the control unit 104 may be arranged to actuate an excitation source or frequency scanner 230 to measure the transducer operating frequency by energizing the transducer 11 at a low power level while scanning the excitation frequency over a pre-determined range of frequencies for example 8.5 Mhz 10.5 Mhz and monitoring the response of the transducer to such excitation and to select the optimal operating frequency.

The ultrasonic system may be similar to that disclosed in U.S. Patent Publication No. 20160008636, entitled “Ultrasound Imaging Sheath and Associated Method for Guided Percutaneous Trans-Catheter Therapy,” the disclosure of which is incorporated by reference herein. Other ultrasonic systems can be utilized.

After preparation of a tracheal access site (or other site), and connecting the catheter 10 to the ultrasound system, the ultrasound catheter 10 is inserted into the working channel of the bronchoscope 5 after the bronchoscope has been advanced to a desired treatment site in a third generation or fourth generation bronchus under visual guidance through the bronchoscope camera or optical fiber. Alternatively, a steerable sheath can be used for catheter delivery, the catheter can be delivered over a guidewire after removal of the scope or the catheter can have a steerable mechanism for advancement directly to the treatment site.

Once the distal end of the catheter is in position within a bronchial branch, pump(s) 216 brings balloon 12 to an inflated condition as depicted in FIGS. 2 and 3. Circumferential contact will be ensured through analysis of a volumetric A mode signal. In this condition, the compliant balloon 12 engages the bronchial wall. Control unit 104 then actuates drive motors 222 via motor control devices 220 to operate pump(s) 216 and also actuates valve(s) 224 to direct saline solution through a lumen in the catheter 10 into a distal end portion of the bronchial tree. The inflated balloon 12 maintains the saline solution in the distal end portion of the bronchial tree during the application of ultrasound through the trapped liquid. Where the transducer 11 inside the balloon 12 is used to treat emphysematous tissue proximate and just outside a bronchial wall, the fluid-filled balloon not only provides for a relatively homogeneous energy distribution circumferentially, but also keeps the very high energy levels close to the transducer located inside the cooling fluid where they are harmless, since ultrasound does not interact with fluid. If these peak energy levels were allowed to be located close to the bronchial wall, injury would result. The other advantage of proper centering is that the treatment volume is coinciding with the relatively flat portion of the 1/r curve providing an almost constant power level throughout the treatment volume and therewith allows for a wide range of treatment depth that can encompass not only very shallow smooth muscle or goblet cell ablation but also more remotely disposed emphysematous tissue for remodeling alveolar structures of distal lung sections.

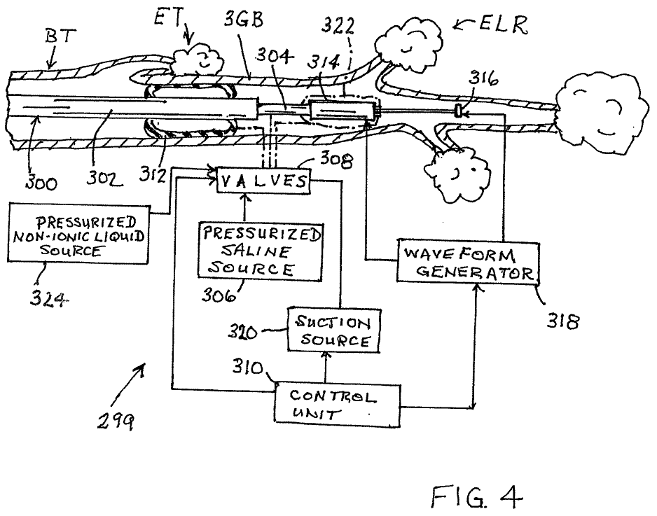

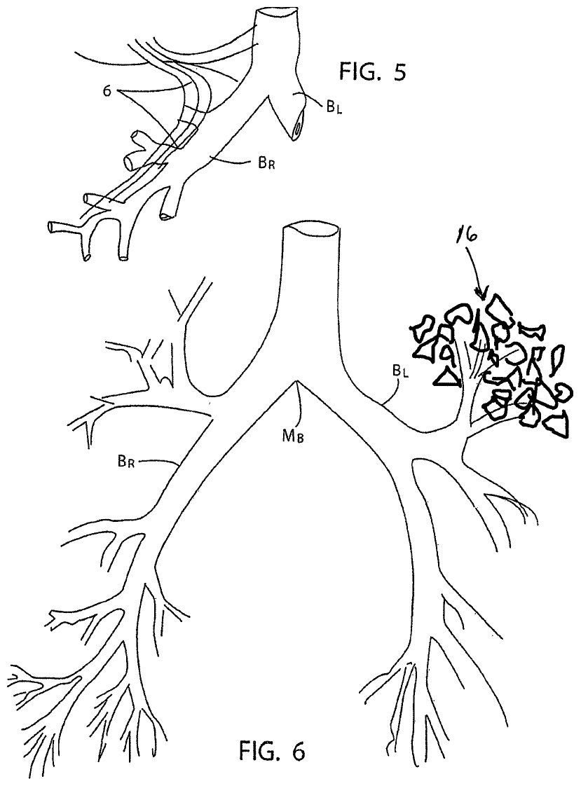

As illustrated in FIG. 4, a system 299 for treating emphysematous tissue comprises an elongate introducer instrument 300 including a distal portion 302 insertable into a bronchial tree BT of a subject diagnosed as having at least one emphysema-damaged lung region ELR. Distal end portion 302 is configured particularly for disposition in a third or fourth generation bronchus 3GB proximate to or in emphysema-damaged lung region ELR. A catheter (tubular member) 304 extends through a lumen of elongate introducer instrument 300 and is slidable relative to the introducer instrument. Catheter 304 is operatively connected at a proximal end to a pressurized source 306 of saline solution controlled via one or more valves 308. An actuator or control unit 310 is connected to valves 308 to effect selective opening of the valves for enabling the introduction of saline solution from source 306 into bronchial tree BT.

The treatment system 299 further comprises an occlusive member such as a balloon 312 attached to an outer surface of introducer instrument 300 proximate a distal end (not separately enumerated) of the introducer instrument. The balloon 312 is expandable to occlude the host bronchial branch (specifically a third or fourth generation bronchus 3GB) to trap saline injected via catheter 304 into a distal section of the bronchial tree BT including the emphysema-damaged lung region ELR, preventing saline from flowing proximally thereof. Thereafter, the clinician inserts into the saline-filled distal section of the bronchial tree BT, via the elongate introducer instrument 300, an acoustic and/or ultrasound transducer in the form of one or more coaxial and longitudinally arranged cylindrical members 314 contained in a non-compliant, or limitedly expandable, cooling balloon 322 supported on catheter 304. Alternatively or additionally, or one or more planar piezoelectric elements 316, optionally in a respective non-compliant balloon (not illustrated), illustratively positioned distal of balloon 322, may be inserted into the saline-filled distal section of the bronchial tree BT. The transducer-containing balloons such as balloon 322 are controllably filled with a circulating pressurized or pressurizable non-ionic liquid from a source 324 via valves 308 for cooling and balloon size control. Once disposed in wave-transmitting contact with the saline solution trapped in the section of the bronchial tree BT (due to the inflated occluding balloon), the acoustic and/or ultrasound transducer 314, 316 is energized by a waveform produced by an electrical waveform generator 318 in response to signals from actuator or control unit 310. The acoustic and/or ultrasound transducer 314, 316 is thereby activated to emit acoustic and/or ultrasound energy into the sealed or trapped saline liquid at a therapeutic level sufficient to damage emphysematous tissue and initiate autogenic remodeling of the emphysematous tissue.

Actuator or control unit 310 may include programming or solid-state circuits and AI configured to additionally activate or energize the transducer 314, 316 with pulses of duration, frequency, and inter-pulse spacing to pulse the acoustic and/or ultrasound energy so as to induce mechanical stress in the emphysematous tissue and thereby break up or modify emphysematous damaged alveoli.

Actuator or control unit 310 may be further configured to select pulse duration, frequency and inter-pulse spacing. To this end, actuator or control unit 310 is configured to test the flooded bronchial section by (i) activating or energizing the transducer 314, 316 with low-power pulses, (ii) processing incoming ultrasonic echoes to determine emphysematous tissue structure, and (iii) selecting parameters of pulse duration, frequency, and inter-pulse spacing in accordance with the determined average or collective alveoli size. Such parameter selection can also be based on AI analysis.

During use of the apparatus of FIG. 1 to treat emphysematous tissue ET (see FIG. 4) proximate and just outside a bronchial wall (e.g., 3GB in FIG. 4) rather than at the most distal regions of the bronchial tree BT, the circulation device 212 (FIG. 1) maintains a flow of cooled non-ionic liquid into and out of balloon 12 (or 322), to cool the transducer 11 (or 314). The cooled balloon also tends to cool the interior surface of the bronchus. Typically, the treatment site is in a third or fourth generation bronchus 3GB proximate emphysematous tissue ET. The liquid flowing within the balloon 12/322 may include a radiographic contrast agent to aid in visualization of the balloon and verification of proper placement fluoroscopically.

The physician initiates the treatment through a user interface. In the treatment, the ultrasonic system, and particularly the control unit 104 and ultrasonic signal or waveform generator 106, actuates transducer 11 (or 314) to deliver therapeutically effective ultrasonic waves to an impact volume 13 (FIG. 2). The ultrasound energy transmitted by the transducer 11 propagates generally radially outwardly and away from the transducer 11 encompassing a full circle, or 360° of arc about the proximal-to-distal axis of the transducer 11 and the axis of the tertiary or fourth generation bronchial section 3GB treated.

The selected operating frequency, unfocused characteristic, placement, size, and the shape of the ultrasound transducer 11 (or 314) enables a “near field” impact region or volume that encompasses smooth muscle, nerves, goblet cells and nearby emphysematous tissue. As shown in FIG. 2 within this region, an outwardly spreading, unfocused omnidirectional (360°) cylindrical beam of ultrasound waves generated by the transducer 11 tends to remain collimated and has an axial length approximately equal to the axial length of the transducer 11. For a cylindrical transducer, the radial extent of the near field region is defined by the expression L2/l , where Lis the axial length of the transducer 11 and 1 is the wavelength of the ultrasound waves. At distances from the transducer surface greater than L2/l, the beam begins to spread axially to a substantial extent. However, for distances less than L2/1l , the beam does not spread axially to any substantial extent (FIG. 2). Therefore, within the near field region, at distances less than L2/l , the intensity of the ultrasound energy decreases according 1/r as the unfocused beam spreads radially. As used in this disclosure, the term “unfocused” refers to a beam, which does not increase in intensity in the direction of propagation of the beam away from transducer 11. These principles, along with other principles and functions of transducer, can also apply to transducer 314.

The impact volume 13 is generally cylindrical and coaxial with the bronchial section treated (FIG. 2). It extends from the transducer surface to an outer impact radius, where the intensity of the ultrasonic energy is too small to heat tissue. Within impact zone 13, the tissue is heated to temperature ranges that will cause inactivation of nerves, smooth muscle-or goblet cell-ablation and, pursuant to the present apparatus and related method, induce remodeling of emphysematous tissue lying near the wall of a third or fourth generation bronchus, see FIG. 7.

As discussed above, the length of the transducer may vary between about 2 mm and about 10 mm but is preferably about 6 mm to provide a wide inactivation zone of the bronchial nerves, smooth muscle, goblet cells and especially emphysematous tissue. The diameter of the transducer may vary between about 1.5 mm to about 3.0 mm and is preferably about 2.0 mm. The dosage is selected not only for its therapeutic effect, but also to allow the radius of the impact volume 13 to be optimized for its therapeutic application without transmitting damaging ultrasound energy to collateral structures like esophagus 3 in FIG. 1.

Operation of the transducer thus provides a therapeutic dosage, which inactivates nerves or ablates smooth muscle, goblet cells or remodels radially proximate emphysematous tissue without causing further damage to the third or fourth generation bronchus 1 and particularly the mucosa. In addition, the circulation of cooled liquid through balloon 12 (or 312) containing transducer 11 (or 314) may also help reduce the heat being transferred from transducer 11/314 to the inner layer of the bronchus. Hence, the transmitted therapeutic unfocused ultrasound energy does not damage the inner layer of the bronchus, providing a safe treatment.

To generate the therapeutic dosage of ultrasound energy for inducing remodeling of sufficiently proximate emphysematous tissue, the acoustic power output of the transducer typically is approximately 10 watts to approximately 100 watts, more typically approximately 20 to approximately 30 watts. The duration of power application typically is approximately 2 seconds to approximately a minute or more, more typically approximately 10 seconds to approximately 60 seconds, see FIG. 7.

For emphysematous tissue remodeling using the apparatus of FIG. 1 or that of FIG. 4, not only heat but also pulsed operation can be used, the latter to cause mechanical tissue interaction. In the affected emphysematous lung regions, pulsed ultrasound is used to create shockwaves that cause mechanical stress through cavitation and therewith break up or remodel endothelial thickening and or trigger healing processes in the alveoli. The optimum dosage used with a particular system to achieve the desired therapeutic effects may be determined by mathematical modeling and confirmed by animal testing. It can also be optimized by AI analysis during treatment.

Numerous variations and combinations of the features discussed above can be utilized. For example, the ultrasound system may control the transducer to transmit ultrasound energy in a pulsed function rather than a continuous function during application of therapeutic ultrasonic energy. Ultrasound transducer may emit ultrasound energy at a duty cycle of, for example, 50%. Pulse modulation of the ultrasound energy is helpful in limiting the tissue temperature while increasing treatment times. The pulsed therapeutic function can also be interleaved with a diagnostic volumetric A mode acquisition. This way, diagnostic ultrasound information can be obtained (quasi)simultaneously to the therapeutic treatment. The pulsed function will also be utilized to create mechanical cavitation effects advantageous to remodel emphysematous tissue within the treatment volume.

In a further variant, the bronchial diameters can be measured by techniques other than actuation of the transducer as, for example, by radiographic imaging or magnetic resonance imaging or use of a separate ultrasonic measuring catheter. In this instance, the data from the separate measurement can be used to set the dose.

In a further variant, the balloon 12 (322) may be formed from a porous membrane or include holes, such that cooled liquid being circulated within the balloon may escape or be ejected from the balloon against the bronchial walls to improve acoustic contact and axial moveability.

Typically, catheter 10 is a disposable, single-use device. The catheter 10 or ultrasonic system may contain a safety device that inhibits the reuse of the catheter 10 after a single use. Such safety devices per se are known in the art.

It is to be understood that in treatments pursuant to the present invention, no foreign body is left in the airway or bronchial system that would decrease the risk of failure durability of the treatment for emphysema and also the complications of granulation tissue formation and also subsequent pneumonia or even empyema. For emphysema, the treatment can be done at the segmental level-decrease the risk of pneumothorax and also allows one to target portions of the diseased lobe therefore saving more viable tissue. The clinician can repeat the invention procedure for all therapies, including denervation, emphysema, and also asthma.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

Additionally, persons skilled in the art will understand that the elements and features shown or described in connection with one embodiment may be combined with those of another embodiment without departing from the scope of the present invention and will appreciate further features and advantages of the presently disclosed subject matter based on the description provided.

Throughout the present invention, terms such as “approximately,” “generally,” “substantially,” and the like should be understood to allow for variations in any numerical range or concept with which they are associated. For example, it is intended that the use of terms such as “approximately” and “generally” and “substantially” should be understood to encompass variations on the order of 25%, or to allow for manufacturing tolerances and/or deviations in design.

Although terms such as “first,” “second,” “third,” etc., may be used herein to describe various operations, elements, components, regions, and/or sections, these operations, elements, components, regions, and/or sections should not be limited by the use of these terms in that these terms are used to distinguish one operation, element, component, region, or section from another. Thus, unless expressly stated otherwise, a first operation, element, component, region, or section could be termed a second operation, element, component, region, or section without departing from the scope of the present disclosure.

Each and every claim is incorporated as further disclosure into the specification and represents embodiments of the present disclosure. Also, the phrases “at least one of A, B, and C” and “A and/or B and/or C” should each be interpreted to include only A, only B, only C, or any combination of A, B, and C.

1. System for pulmonary treatment, comprising:
an ultrasound transducer adapted for insertion into the bronchial tree of the mammalian subject and for transmitting circumferential ultrasound energy; and
an actuator or control unit electrically connected to the transducer and adapted to control the ultrasound transducer to transmit ultrasound energy into an impact volume encompassing a bronchial branch so that the ultrasound energy is applied at a therapeutic level sufficient to a) inactivate conduction of bronchial nerves and to b) ablate smooth muscle, ablate goblet cells, and/or remodel emphysematous tissue throughout the impact volume.

2. The system of claim 1, wherein the actuator or control unit is configured to energize the transducer, to process ultrasonic echo encoding electrical signals from the transducer, to generate volumetric A mode signal data from such signals and to make a volumetric A mode bronchial diameter measurement from the volumetric A mode signal data.

3. (canceled)

4. The system of claim 1, wherein the actuator or control unit is adapted to control activation or energization of the transducer based on the volumetric A mode measurement and optimized for each individual treatment site and the surrounding coupling fluid temperature as well as pre-cooling time so as to maintain the temperature of the bronchial wall below 40° C. while achieving a temperature above 60° C. throughout the impact volume surrounding the bronchial branch at the individual treatment site.

5. The system of claim 1, wherein the actuator or control unit transmits ultrasound energy in the diagnostic mode to generate a signal and integrate ultrasound echoes to measure bronchial diameter.

6. The system of claim 1, wherein the actuator or control unit is configured to transmit ultrasound energy at a subtherapeutic level in a diagnostic mode to provide centering of the transducer in the bronchial branch.

7. The system of claim 1, wherein the actuator or control unit is adapted to control the ultrasound transducer to transmit the ultrasound energy in a pulsed function interleaved with volumetric A mode diagnostic acquisitions, thereby operating in a quasi-simultaneous therapeutic/diagnostic mode.

8. The system of claim 1, wherein the actuator or control unit is adapted to control the ultrasound transducer to transmit the ultrasound energy in pulses to mechanically remodel emphysematous tissue by inducing mechanical stress in the affected lung segments to cause fibrosis.

9. The system of claim 1, further comprising an occluding balloon expandable to prevent backflow of liquid injected into a distal section of the bronchial tree.

10. The system of claim 9, further comprising an elongated member supporting the transducer, wherein the elongated member is introduced through an introducer, and the introducer supports the occluding balloon.

11. The system of claim 1, further comprising a catheter with a lumen operatively connectable to a source of pressurized or pressurizable saline liquid to flood a distal emphysematous section of the bronchial tree with saline liquid in order to ensure ultrasound conduction and thermal and mechanical interaction throughout a treatment volume within or proximate the bronchial branch.

12. The system of claim 11, wherein the lumen is connectable to a suction source to apply a vacuum to a distal emphysematous lung region to evacuate fluids and mucous.

13. The system of claim 1, further comprising an elongated member with a distal end and a proximal end, the transducer being mounted to the elongated member adjacent the distal end inside a balloon.

14. The system of claim 13, wherein the balloon is filled with circulating fluid, in order to cool, center, align and acoustically couple the transducer with a wall of the bronchial branch, the balloon includes a porous membrane to engage the wall of the bronchial branch with fluid for improved acoustic coupling while enabling moving the balloon axially to achieve optimal positioning based on volumetric A mode signal analysis of emphysematous tissue.

15. (canceled)

16. The system of claim 1, wherein the actuator or control unit is further adapted to process volumetric A mode signals to determine depth of emphysematous tissue and to optimize the ultrasound ablation energy and cooling temperature for the determined depth.

17. The system of claim 1, further comprising an elongated member with a distal end and a proximal end, the transducer being disposed inside a compliant balloon mounted to the distal end of the elongated member and filled with circulating transducer-cooling and ultrasound-coupling fluid, wherein the actuator or control unit is configured to control the ultrasound transducer to vary acoustic power and transducer-activation duration and to vary temperature of the circulating transducer-cooling and ultrasound-coupling fluid to optimize, in accordance with volumetric A mode determined size of the bronchial branch, therapeutic effectiveness of the ultrasound energy generated in the impact volume and target nerves, smooth muscle, goblet cells and/or or emphysematous tissue to be treated.

18. The system of claim 1, wherein the system further comprises a catheter for supporting the transducer and an introducer having a lumen for passage of the catheter, the catheter having a first balloon containing the transducer and the introducer having a second balloon proximal the first balloon to prevent flow of fluid proximal the second balloon.

19. The system of claim 18, wherein the introducer or catheter has a lumen for passage of the fluid into a distal section of the bronchial tree for ultrasound conduction.

20. (canceled)

21. The system of claim 13, wherein the balloon is filled with a cooling fluid and the actuator or control unit maintains a temperature of the cooling fluid at or below body temperature.

22. (canceled)

23. The system of claim 18, further comprising a pressure sensor to monitor pressure to enable the second balloon to be maintained in an expanded occluding condition.

24. The system of claim 18, wherein the fluid floods the distal section, and the actuator or control unit is configured to test the flooded section.

25-61. (canceled)
